(19) United States
(12) Patent Application Publication
(10) Pub. No.: US 2006/0128665 A1
(43) Pub. Date: Jun. 15, 2006

(54) Marine lipid compositions

(75) Inventors: Steve Leigh, Amsterdam (NL); Elsa Kung, Basel (CH); Peter Van Hoogevest, Bubendorf (CH); Henricus Tiemessen, Weil am Rhein (DE)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: PHARES PHARMACEUTICAL RESEARCH N.V., Curracao (NL)

(21) Appl. No.: 10/536,450

(22) PCT Filed: Nov. 26, 2003
(86) PCT No.: PCT/EP03/13299

§ 371 Date: Oct. 28, 2005

(30) Foreign Application Priority Data
Nov. 26, 2002 (EP) 02258116.9
Aug. 25, 2003 (EP) 03255278.8

(51) Int. Cl. A61K 31/685; A61K 31/66; C07F 9/02

(52) U.S. Cl. 514/78; 514/120; 554/78

(57) ABSTRACT

There is described marine phospholipids (MPL) compositions suitable for human, aquaculture applications comprising a dry composition comprising nutritional components selected from the group consisting of marine phospholipids, marine proteins and amino acid blends obtainable by fluid extraction of a dried marine raw material. The compositions have low amounts of neutral lipids and particularly low amounts of cholesterol and cholesterol esters. They may be used either as such in powder form and for preparing purified MPL compositions by ethanol extraction. Both forms may be used as nutritional supplements, ingredients for functional foods and to optimise the delivery and containment of nutritional components in multicomponent fish feed compositions. There is further described a blend of the purified, ethanol extracted MPL compositions mixed with marine, vegetable, microbial or MCT oils to form liquid preparations for hard or soft gel encapsulation. There is further described MPL concentrates comprising purified and clearly defined phospholipid types dispersed in hydrophilic medium which are nutritionally essential in larvae feed. Still further there is described MPL compositions in unit dosage forms as supplements to supply highly bioavailable omega-3 fatty acids for human use, optionally with biologically active compounds.

This invention relates to marine phospholipid compositions (MPL) comprising long chain omega-3-fatty acids. The compositions are particularly suitable as supplements for human consumption, animal feed or for nutritional and embedment purposes in feed compositions for aquatic livestock and fish larvae.

### BACKGROUND OF THE INVENTION

Phospholipids are amphipathic lipids which are components of all cell membranes. The phospholipid molecule comprises a phosphoric acid ester head group attached to a glycerol backbone with one or more usually two fatty acid chains. The head group may be neutral, anionic, cationic or zwitterionic depending on the pH. The different types of phospholipids are identified by their head groups. Regardless of the source, the most widely occurring natural phospholipid is phosphatidylcholine (PC) which has choline as a head group. The second most abundant is phosphatidylethanolamine (PE). Phosphatidyl inositol (P), phosphatidyl serine (PS), sphingomyelin (SPM) and the monoacyl derivatives are usually found in smaller amounts. The chief difference between the phospholipids from different sources is reflected in the fatty add profiles which vary according to chain length and degree of unsaturation.

The fatty acid moieties attached to phospholipids from natural sources have 14 to 24 carbon atoms. The unsaturated fatty acids mostly comprise C18 to C22 chains and may contain between one to six double bonds depending on the origin, i.e. marine, animal, or plant. Plant derived phospholipids usually do not contain fatty acids with a chain length of more than 18 carbon atoms. As an example about 65 wt % to 75 wt % of the fatty acids in soya (mainly C18) comprise one to three double bonds, approximately 60 wt % of which is the omega-6 linoleic acid with two double bonds. In comparison, egg phospholipids additionally comprise some longer chain fatty acids (C20 and C22 with four and 6 double bonds respectively). Marine phospholipids are characterised by very high levels of mostly long chain C 20 and C22 highly unsaturated fatty acids (HUFA), eicosapentaenoic acid (EPA) and docosahexaenoic CONFIRMATION COPY acid (DHA). The striking feature of marine phospholipid fatty acids is the prevailing presence of a double bond in the omega-3 position of the fatty acid chain.

Soya and egg phospholipids are widely employed for their ubiquitous and multi-functional properties. They are used extensively in all types of applications in the food and feed industries particularly as emulsifiers because of their amphiphilic nature. They are also employed as a rich source of unsaturated fatty adds such as linoleic, linolenic (soya), arachidonic and docosahexaenoic acid (egg) in nutritional and functional foods. Phospholipids from plants do not contain the type of long chain highly unsaturated omega-3-fatty acids found in fish oils. The level of HUFAs in marine phospholipids is higher than in the corresponding oily triglycerides from the same source. Furthermore the bioavailability of the fatty acids is believed to be higher from phospholipids compared to triglycerides. This may be due to the amphiphilic properties of phospholipids reflected in better water dispersibility and/or their greater susceptibility to phospholipases compared to the glycerolysis of triglycerides. Thus marine phospholipids would appear to offer an extremely rich source of long chain omega-3 polyunsaturated fatty adds for incorporation into fish diets and as supplements for humans.

The prior art relating to marine phospholipids in aquaculture is chiefly concerned with preparing fish feed compositions from different marine sources and raw materials or biomass.

WO 00/27218 describes a composite dry feed for fish larvae to replace live feed comprising a matrix containing water-insoluble nutrients and phospholipids embedded in the particles and a method of preparing the feed particles.

WO 01/50884 describes a composite feed for feeding prey organisms suitable for aquaculture. The lipid component is derived from marine organisms such as fishmeal, phytoplankton or zoo plankton biomass.

EP 0 996 740 provides a particulate material with high proportion of DHA in the lipid fraction and a mean particle size between 5 microns to 10 microns, suitable as a nutritional supplement in aquaculture. The method describes a protein/phospholipid composition extracted from an aqueous suspension of broken algal cells using solvent extraction and removal of water by spray drying.

Feed compositions for normal fish diets employ phospholipid mixtures (lecithin) from soya to emulsify lipophilic and hydrophilic components and hold them together in the feed particles. However, in special balanced compositions for larvae start up feed and weaning, it is essential that well characterised phospholipids are used. The high percentage of esterified polyunsaturated fatty acids, the bio-membrane forming properties on top of the emulsifying properties, make marine phospholipids more effective to optimise nutritional properties and minimise leakage, particularly of water soluble nutrients. Compositions comprising selected marine phospholipids and other components can form versatile lipid aggregates which will entrap both water soluble and oil soluble compounds either by association or solubilisation in addition to emulsification. The entrapment depends on the type of aggregates, which may be bilayered or miceuar, vesicular or non vesicular, according to the composition of the phospholipid mixture. Liposomes are one example of vesicular structures whilst micro emulsion droplets, micelles and mixed micelles are examples of non vesicular structures useful for entrapment. The outstanding feature of lipid aggregates derived from the MPL compositions described in this invention is their unexpected capacity for containment of both hydrophilic and lipophilic materials not only in human nutritional supplements and functional foods, but also in animal, fish and larvae feed compositions particularly in the presence of a destabilising medium like sea water.

### SUMMARY OF THE INVENTION

The invention is in the area of ‘particulate’ and ‘waxy ’ marine phospholipid compositions and ‘HUFA concentrates’ suitable as supplements in human nutrition and functional food and also for embedment and nutritional purposes in animal and fish feed microparticle compositions.

A) Particulate Marine Phospholipid/Marine Protein and Amino Acid Blend

The present invention relates to a marine phospholipid (MPL) composition suitable for human or aquaculture applications comprising a dry composition, i.e. in particulate or powdered form, comprising nutritional components selected from the group consisting of marine phospholipids, marine proteins, amino acids, minerals prepared by solvent or gas extraction under super or hypercritical conditions of a dried marine raw material comprising a major amount of polar and a lower amount of non polar (neutral) lipids. The dried marine raw material has low moisture content i.e. below 10 wt %.

The composition comprises,

I). 10 wt % to 30 wt % total (polar and neutral) lipids;

II) 70 wt % to 90 wt % marine proteins and amino acid blends. 70 wt % to 95 wt % of the total lipids in I) consist of polar lipids and 5 wt % to 30 wt % consist of neutral lipids.

Preferably, the composition comprises,

I) 15 wt % to 30 wt % total (polar and neutral) lipids;

II) 70 wt % to 85 wt % marine proteins and amino acid blends.

Preferably 80 wt % to 95 wt % of the total lipids in I) consist of polar lipids and 5 wt % to 20 wt % consist of neutral lipids.

The invention also describes a particulate composition wherein 40 wt % to 80 wt % of the polar lipids consist of phosphatidylcholine or mixtures of PC and the monoacyl derivative thereof, wherein said phosphatidylcholine and the monoacyl derivative are esterified with 30 wt % to 60 wt % HUFA's. The balance of 20 wt % to 60 wt % of the polar lipids in the composition contain phospholipids selected from the group consisting of phosphatidyl ethanolamine (PE), phosphatidyl inositol (PI), phosphatidyl serine (PS), sphingomyelin (SPM) and the monoacyl derivatives thereof.

B) Purified, Waxy Marine Phospholipid (MPL) Composition

The invention further describes a purified, viscous or paste-like, waxy MPL composition prepared by ethanol extraction from a particulate MPL composition. The waxy MPL composition contains,
i) 70 wt % to 95 wt % or more of total lipids,
ii) 5 wt % to 30 wt % of neutral lipids.

The purified waxy MPL composition may be prepared by ethanol extraction of a dried composition comprising nutritional components selected from the group consisting of phospholipid/marine protein/amino acid/minerals blend.

The composition preferably contains not more than 15 wt % neutral lipids and at least 40 wt % of phosphatidylcholine or mixture of PC and the monoacyl derivative thereof, calculated on the basis of all the phospholipids, wherein at least 30 wt % of the fatty acids are omega-3 fatty acids esterified to the phospholipids.

The resulting waxy composition may be further blended with fish or vegetable oils to prepare a liquid composition that may be filled into hard or soft gelatine capsules or the like, as a supplement for human nutrition.

C) Water Dispersible Marine Phospholipid (MPL) Concentrate

The invention further provides water-dispersible marine phospholipid (MPL) concentrate in hydrophilic medium which may be prepared from a waxy composition for use in multicomponent fish and larvae feed compositions referred to as ‘microparticles’ for optimising the containment and retention of oil soluble and water soluble nutritional components.

The embodiment includes a homogeneous water-dispersible concentrate which comprises:

i) 25 wt % to 75 wt % of a marine phospholipid composition comprising PC or mixtures of PC and the monoacyl derivative in an amount of at least 40 wt % as the major component and minor amounts of a phospholipid selected from the group consisting of PE, PI, SPM and PS and their monoacyl derivatives,

ii) 15 wt % to 75 wt % of ethanol or at least one polyol or mixtures thereof,

iii) water to make 100%; and, optionally, further additives selected from the group consisting of polymers, nutritional components, stabilisers, preservatives, and antioxidants.

The invention also includes a method of preparing lipid aggregates for embedment in fish and larvae feed micro particles which comprises dispersing in water a lipid concentrate comprising,

i) 25 wt % to 75 wt % of a marine phospholipid composition comprising PC or mixtures of PC and the monoacyl derivative in an amount of at least 40 wt % as the major component and minor amounts of at least one phospholipid selected from the group consisting of PE, PI, SPM and PS and their monoacyl derivatives,

ii) 15 wt % to 75 wt % of ethanol or at least one polyolor mixtures thereof,

iii) water to make 100%, and, optionally,

iv) further additives selected from the group consisting of polymers, nutritional components, stabilisers, preservatives, and antioxidants;

In humans, MPLs are particularly useful as supplements to prevent coronary heart disease (CHD), to reduce elevated blood cholesterol and triglycerides, high blood pressure, high blood glucose and to treat several mental disorders (such as Alzheimer), premenstrual syndrome (PMS), inflammatory bowel disease, osteoarthritis, inflammatory skin diseases etc.

Omega-3 fatty acids also need to be supplied in aquaculture diets because of the inability of fish larvae to synthesise these compounds de novo from shorter chain precursors. Lack of these essential fatty acids impair growth levels.

The invention includes marine phospholipid blends which may be dry or waxy compositions and water dispersible concentrates in admixture with phospholipids selected from the group consisting of vegetable or egg phospholipids, enzyme modified phospholipids and synthetic and semi-synthetic phospholipids comprising C14 to C22 fatty acids with one to six double bonds, wherein the marine phospholipids preferably comprise the major component i.e. more than 50% by weight of the total mixture. The compositions may further comprise biologically active compounds.

### DETAILED DESCRIPTION OF THE INVENTION

In this specification:

the numerical ranges with regard to the components in the compositons indicate exact and approximate (ca., about) ranges;

‘lecithin’ is used as a broad definition to describe mixtures comprising phosphatidylcholine (PC) and other types of phospholipids. It is not used interchangeably as a narrow definition to describe only PC. It also covers mixtures of phospholipids from different sources and mixtures thereof;

‘marine phospholipid’ (MPL) refers to the phospholipid compositions comprising predominantly long chain and highly unsaturated fatty adds obtained from a ‘marine raw material’ e.g. roe and milt of fish such as salmon, herring, capelin, saithe and cod, etc. It includes phospholipids from all forms of marine vegetation and life forms such as krill and other crustaceans and those prepared by synthesis or partial synthesis (e.g. enzymatic fatty acid exchange) involving phospholipids from other sources and different species. The term also extends to blends of marine phospholipids and egg or soya phospholipids with the said MPL as the major component in the mixture. The phospholipid content may vary by up to +/−25 wt % of the values normally quoted for different species depending on origin, source, climate and other seasonal factors. The definition also includes mixtures or blends comprising marine diacyl phospholipids and the monoacyl derivatives prepared by enzyme hydrolysis using phospholipase A2 on a suitable marine phospholipid substrate.

‘polar lipids’ are amphipatic lipids such as phospholipids, glycolipids and sphingolipids which are components of cell membranes from marine organisms;

‘neutral lipids’ are non polar lipids such as mono, di and triglycerides, free fatty acids and esters, cholesterol, cholesterol esters and carotenoids which are present in marine organisms;

‘total lipids’ are combined polar and neutral lipids; “marine proteins and amino acids” refers to proteinaceous materials originating from marine organisms;

‘concentrate’ describes a standardised and defined mixture prepared from purified MPL comprising HUFAs, predominantly DHA and EPA in a water dispersible hydrophilic medium. The concentrate may comprise a mixture of marine phospholipids blended with soya, egg, synthetic, semisynthetic, and enzyme modified phospholipids. It may be a liquid or gel like composition suitable for preparing multicomponent feed for aquatic lifestock including microparticles for fish larvae and weaning. It may also be administered as supplement for human use as such or in a unit dosage form.

‘nutritional components’ include all substances which are nutritionally valuable to humans, livestock, fish larvae, smelt and other marine species. Examples include phospholipids, proteins, amino acids, minerals, vitamins, fish protein hydrolysates, fish oil triglycerides and carbohydrates.

‘long chain fatty acids’ refer to fatty adds with more than 18 carbon atoms starting with eicosa (C20) and docosa (C22) fatty acids;

‘highly unsaturated’ fatty acids (HUFA) may have three or more usually four or more double bonds, e.g., tetraenoic(4), pentaenoic(5) and hexaenoic(6) which are either omega-6 or omega-3fatty acids. Eicosatetraenoic acid (AA) is an omega-6 fatty acid, eicosapentaenoic acid (EPA) and docosahexaenoic acid (DHA) are omega-3 fatty acids;

‘fluid extraction’ means ‘de-oiling’ or removal of neutral lipids from a dried marine raw material using a solvent in liquid form, such as acetone, or a supercritical gas such as carbon dioxide, nitrous oxide or propane or mixtures thereof, under hypercritical (supercritical) conditions;

‘ethanol extraction’ describes the purification of a dry MPL composition after deoiling by using ethanol or related solvents such as methanol, isopropanol, tert-butanol, n-propanol and mixtures thereof to extract the polar lipids from proteins and amino acids and other impurities. A waxy MPL composition is obtained after removal of the ethanol or related solvent.

‘particulate composition’ describes a dried, powdered composition with average particle diameter above 0.5 mm with moisture content that is below 10%.

Preparation of Marine Phospholipid (MPL) Compositions

Standard procedures to obtain purified or semi-purified phospholipids particularly from soya and egg are based on a multi-step fractionation and/or purification process starting from crude, commercial lecithins.

Removal of Lecithin from Crude Vegetable Oils (Degumming)

Vegetable lecithins may be obtained by simple water degumming of crude oils or by other processes like specific centrifugation of the wet starting materials. However, because of the variable contents and compositions of MPLs from different origins, it is not very satisfactory to utilise currently available procedures based on marine raw materials and biomass with high water content to prepare marine phospholipids for human nutrition and fish feed compositions without modification of the process. Improved extraction methods are therefore required for preparing MPL compositions with desirable amounts of polar lipids and low amounts of neutral lipids which are preferred in human nutriceutical and functional food applications and for forming lipid aggregates embedded in feed microparticles.

Drying

Drying using elevated temperatures or spray drying is usually employed to prepare MPL containing powders from crude, aqueous fish roe and milt, or biomass from broken marine algal cells containing large amounts of water. In this invention it is preferable to use alternative technologies, for example freeze drying or other low stress drying methods to dry the marine raw materials so that they have lower amounts of water prior to processing.

Deoiling

Preferred methods for the removal of neutral lipids, such as triglycerides, cholesterol and cholesterol esters, from the dry marine raw material are, fluid extraction with suitable liquids for instance acetone, and gases like carbon dioxide or other suitable gases under supercritical conditions.

The product remaining after acetone or more preferably CO₂ extraction is a particulate composition comprising protein, amino acids, minerals, polar lipids and smaller (low) amounts of neutral lipids. The particulate MPL composition comprises 70 wt % to 90 wt % of marine protein and amino acids, preferably below 80 wt %. The polar and neutral lipids amount to 10 wt % to 30 wt %, preferably above 15 wt %. The polar lipids amount to 70 wt % to 95% wt %, preferably above 80 wt % of the total lipids whilst the neutral lipids make up the balance amounting to 5 wt % to 20 wt %, preferably below 15 wt %. The moisture content is below 10 wt % whilst minerals and trace elements based on ash residues make up less than 10 wt %.

The composition may be used as such, in powder form in nutritional supplements, in unit dosage form, or for functional food applications, on its own or in combination with biologically active compounds. It may also be used as a supplement in fish feed microparticles. Typically, the composition is a free flowing powder.

Removal of Proteins

The deoiled MPL powder may be further processed and purified using e.g. ethanol extraction to remove t the phospholipids and leave behind proteins, amino acids and most minerals as by products. Besides ethanol, other solvents such as methanol isopropanol, tert-butanol and n-propanol are equally suitable.

After removal of the ethanol or another suitable solvent by evaporation, the composition is a waxy, paste like material and comprises a purified MPL composition containing 70 wt % to 95 wt % (or more after column chroatography) of phospholipids, preferably above 75 wt % with a minimum PC content of 40 wt %, preferably between 60 wt % to 80 wt % as the major component. About 30 wt %, preferably above 40 wt % of the esterified fatty acids in the PC comprise HUFAS. Preferably the HUFAs comprise between 40 wt % to 60 wt % of the omega-3 fatty acids EPA and DHA. Other phospholipid types and typical amounts present are, PE (15 wt %), PI (10 wt %), SPM (5 wt %), PS (2 wt %) and their mono acyl derivatives (4 wt %). Also smaller amounts of glycolipids may be present.

The non polar (neutral) lipids total 5 wt % to 30 wt % of the waxy MPL composition, preferably below 15 wt %. The maximum amount of cholesterol and cholesterol esters is about 8 wt %, preferably below 5 wt %. The phospholipid values are based on phosphorous-31 NMR determinations. This purified composition may also be used for preparing the MPL concentrates in a liquid form

Further Purification

If higher amounts of PC are required, further purification may be carried out using column chromatography with aluminium oxide or silica to yield PC enriched MPL having between 60 wt % to 95 wt % PC. Aluminium oxide is the material of choice if higher than 80% PC concentration is desired.

It is essential that the MPL compositions provide the desired fatty acid profiles and also allow maximum containment and delivery of nutritional components in a suitable form. The idea behind this invention is to exploit the intrinsic properties of particular types of marine phospholipid in compositions which are nutritionally beneficial and furthermore have the capacity to contain nutritional and other components by association/complex formation.

Factors to be considered in using a particulate MPL composition as nutritional supplement and food additive for human use are,

i) between 70 wt % to 95 wt % of phospholipids in the lipid fraction which contain 10 wt % to 30 wt % of total lipids,

ii) phosphatidylcholine content more than 40 wt %, preferably more than 60 wt % of the phospholipids with at least 30 wt % of HUFAs, wherein the fatty acid profiles provide desired levels of DHA and EPA,

iii) low amounts of neutral lipids, preferably below 15 wt %,

iv) low in cholesterol and cholesterol esters, preferably below 5%,

iv) suitability as solid and conversion to liquid unit dosage forms.

Additional factors which may be taken into account in using a purified waxy MPL composition after fluid extraction for preparing the MPL concentrates suitable for nutritional and embedment purposes in fish feed micro particles are,

i) compatibility with other nutritional components for maximum association,

ii) steric effects of long chain highly unsaturated fatty adds which affect aggregate formation, structure, particle size and stability,

iii) avoidance of more than 50% by weight of phospholipids with esterified C18:2 fatty adds and more saturated fatty acids which may cause increased membrane rigidity,

iv) potential to prepare water dispersible compositions for providing magnum amounts of MPL in feed particles,

v) avoidance of elevated temperatures and intensive mixing which may be deleterious in preparing concentrates that form lipid aggregates in water,

vi) avoidance of organic solvents particularly ethanol which are commonly employed in (v) to solubilise phospholipids.

vii) formation of small, homogeneous and stable lipid aggregates comprising nutritional components and optionally polymers for embedment in the feed microparticles,

viii) industrially applicable method of incorporating into feed compositions such as microparticles comprising excipients such as binders and stabilisers,

ix) stability of the aggregates between pH 5 to pH 8,

x) charge on the head group and effect of divalent ions which may cause fusion of the lipid aggregates,

xi) osmotic shock effects,

xii) microbial contamination and overgrowth.

A)

The invention provides ready to use marine phospholipid (MPL) compositions suitable for human or aquaculture applications which may be a dry composition comprising nutritional components selected from the group consisting of marine phospholipids, marine proteins and amino acid blends obtainable by fluid extraction of a dried marine raw material with less than 50 wt % water, preferably less than 10 wt %.

A typical particulate composition prepared by fluid or supercritical gas extraction of the dry marine raw material comprises:

| | |
|---|---|
| Protein and amino adds: | 70 wt % to 90 wt % |
| Total lipid (polar & non polar) | 10 wt % to 30 wt % |
| Polar lipids: | 70 wt % to 95 wt % |
| PC content: | 40 wt % to 80 wt % (of polar lipids) |
| esterffied HUFAs: | 30 wt % to 40 wt % (of polar lipids) |
| Neutral (non polar) lipids: | 5 wt % to 20 wt % |
| cholesterol, cholesterol esters: | <8 wt % |
| free fatty adds: | <5 wt % |
| Iodine value (of the lipid fraction): | 110-130 |
| Minerals, trace elements: | <8 wt % |
| Water: | <10 wt % |

B)

The invention also describes a method to prepare a purified, waxy marine phospholipid composition from a phospholipid/marine protein/amino acid blend after acetone or more preferably supercritical gas treatment of a dried marine raw material, suitable for direct human consumption or other applications, obtainable by extraction of the phospholipids using solvents such as ethanol, methanol, isoproyl alcohol, tert-butanol and n-propyl alcohol, and mixtures thereof. The compositon obtained is a purified, waxy marine phospholipid mixture with low amounts of neutral lipids suitable for preparing a water dispersible concentrate.

The waxy composition may also be blended or mixed with a neutral oil to prepare a liquid or gel like lipophilic composition which may be used in hard or soft gelatine capsules or the like for human nutritional purposes. The oil may be any physiologically acceptable neutral oil. Preferably it is a fish or a vegetable oil or it may be a medium chain triglyceride such as Miglyol and edible fatty add esters, ethers. The amount of oil for blending may be between 25 wt % to 60 wt % or more depending on the purpose. The oil may simply be a diluent or it may be used as a supplement of unsaturated omega-6 fatty adds.

The waxy MPL composition comprises about 70 wt % to about 95 wt % or more of phospholipids comprising at least 40 wt % phosphatidylcholine calculated on the basis of the total phospholipids present which comprise about 30% by weight of esterfied omega-3 fatty acids and between 5 wt % to 30 wt % of neutral lipids. The purified phospholipid mixture may be preferred for preparing a water dispersible marine phospholipid (MPL) concentrate suitable for embedment in fish feed microparticles or as supplements of highly bioavailable long chain omega-3 fatty acids for human, animal and aquaculture use.

A typical specification of the purified, waxy MPL composition prepared by the method described in this invention comprises:

| | |
|---|---|
| Polar lipids: | 70 wt % to 95 wt % |
| PC content: | 40 wt % to 80 wt % |
| esterified HUFAs: | 30 wt % to 40 wt % of polar lipids |
| non polar (neutral) lipids: | 5.0 wt % to 30 wt % |
| cholesterol, cholesterol esters: | <8 wt % |
| free fatty acids: | <5 wt % |
| Iodine value: | 110-130 |
| ethanol: | <2.5 wt % |
| minerals, trace elements (ash): | <2 wt % |
| water: | <5 wt % |

C)

In a further aspect, the invention describes a homogeneous water dispersible MPL concentrate which comprises:
i) 25 wt % to 75 wt % of a MPL composition comprising PC or blend of PC and the monoacyl derivative prepared by enzyme hydrolysis in an amount of at least 40 wt % as the major component and minor amounts of other components including PB, PI, SPM, PS and their monoacyl derivatives,
ii) 15 wt % to 75 wt % of glycerol or another polyol, or sugar,
iii) 1 wt % to 50 wt % water,
iv) optionally other additives selected from the group consisting of polymers, nutritional components, stabilisers, preservatives, and antioxidants.

The water dispersible concentrate may be utilised in food for human nutrition, or as a supplement administered in a suitable unit dosage form with or without biologically active compounds. It may also be used to prepare lipid aggregates embedded in feed particles to contain nutritional components for feeding fish livestock or fish larvae. Preferably, the water dispersible concentrate is prepared using a purified, waxy phospholipid composition with at least 40% by weight of PC purified by ethanol extraction of a particulate or powdered marine phospholipid/marine protein, amino acid and minerals composition.

The invention further describes a method to prepare a water dispersible MPL concentrate from MPL compositions obtained by fluid and ethanol extraction.

The invention further provides a method of preparing lipid aggregates which involves dispersing a lipid concentrate (B) comprising:
i) 25 wt % to 75 wt % of an MPL composition comprising PC or blend of PC and the monoacyl derivative prepared by enzyme hydrolysis in an amount of at least 40 wt % as the major component and minor amounts of other components, including PE, PI, SPM, PS and their monoacyl derivatives,
ii) 15 wt % to 75 wt % of glycerol or another polyol or sugar
iii) 1 wt % to 50 wt % water,
iv) optionally, other additives selected form the group consisting of polymers, nutritional components, stabilisers, preservatives, and antioxidants;
   in water or an aqueous medium for embedment and inclusion in feed particles or addition to food for human consumption.

The fatty acid profile of the phospholipids is characterised by at least 30 wt % of HUFAs, chiefly DHA and EPA based on the total fatty acid content present. Preferably it should comprise between 40 wt % and 60 wt % omega-3 fatty acids.

Hydrophilic Medium

Suitable hydrophilic medium are non toxic polyols such as glycerol, propylene glycol, polyglycerols and mixtures thereof. The preferred polyol is glycerol. For human use ethanol may be suitable. Alternative polyols which may be used in place of or in addition to glycerol are aqueous sugar solutions comprising at least 25 wt % of e.g, glucose, sucrose, soluble maltodextrins, or polyhydric alcohols like mannitol and sorbitol etc.

Polymers

Natural gums, particularly sodium alginates which can cross link with Ca++ ions are preferred as matrix forming material for the microparticles. Alternative materials are proteins such as gelatine, poly peptides and peptides such as casein and soya proteins.

Stabilisers

These include buffers, osmotic components, antioxidants and anti microbials commonly used in fish feeds and supplements.

For fish feed applications a purified, waxy MPL composition after ethanol reatment, is hydrated in the hydrophilic medium, preferably without using elevated temperatures to prepare a homogeneous water dispersible gel like concentrate. Preferably, permitted antioxidants and preservatives may be added. On mixing with aqueous medium, the concentrates readily disperse into a variety of lipid aggregates below 10μ average diameter, preferably below 1μ measured by laser light diffraction. The concentrate further comprising nutritional components and alginates may be embedded in micro particle feed compositions. The amount of MPL concentrate that may be incorporated and embedded in the multicomponent feed composition may be between 10 wt % to 75 wt %, preferably 20 wt % to 40 wt %. After dehydration or lyophilisation (where appropriate) between 25 wt % to 75 wt % of water soluble nutritional components such as protein hydrolysates based on the total phospholipid content may be retained in the microparticles. The high capacity is due to the unexpected properties of the marine phospholipid concentrates for containment of both water and oil soluble nutritional components in compositions in a destabilising environment like sea water.

It should be understood that the invention also includes MPL compositions wherein the mixture comprises marine diacyl phospholipids and their monoacyl derivatives. The mixture is prepared by enzyme hydrolysis using phospholipase A2 or A1 on a phospholipid substrate which may be either a particulate MPL composition or a waxy composition according to the method described in EP 1011634. The amount of diacyl phospholipid to monoacy phospholipid in the enzyme hydrolysed MPL composition may be between 1:10 to 20:1.

There is described, characterised and defined marine phospholipid (MPL) compositions which may be used either as such in powder form or for preparing purified waxy lipid compositions which may be used to prepare improved water dispersible MPL concentrates, as nutritional supplements and/or ingredients for functional foods as well as the delivery and containment of nutritional components in multicomponent fish feed compositions. The MPL compositions in powder form and as water dispersible concentrates may be used in unit dosage forms as supplements to supply highly bioavailable omega-3 fatty acids for human use. The dosage forms may also contain biologically active compounds in combination with the MPL compositions. There is also described MPL concentrates comprising standardised and dearly defined phospholipids for embedment in feed microparticles, which are nutritionally essential in larvae feed.

### EXAMPLE 1

A dried marine raw material consisting 1000 g of freeze dried capelin roe with a total lipid content of 35 wt % is extracted in a batch extraction vessel with 40 kg/h carbon dioxide at 300 bar and 40° C. for 4 hours to remove 180 g of a clear orange-brown oil containing 18 wt % of cholesterol and cholesterol esters. The particulate MPL composition in the form of a coarse powder which is recovered contains a total lipid content of about 21 wt %, of which the major proportion (about 17 wt %) is polar phospholipids and 4 wt % are neutral lipids. About 70 wt % of the phospholipids is phosphatidylcholine comprising at least 30 wt % HUFAS. About 70 wt % of the composition is proteinaceous material including about 8 wt % minerals and trace elements. The composition has a mild fishy taste and odour with a peroxide value below 3 and may be used as such or mixed with suitable excipients in functional food applications. A unit dose of 300 mg of the powder may be filled into hard gelatine capsules for oral use as nutritional supplement. The powder may also be converted into tablets. Optionally the MPL composition may contain biologically active compounds

In place of capelin roe, herring or cod roe may be used. An alternative dried starting material that may be used in this example is from krill.

### EXAMPLE 2

820 g of the powder composition from Example 1 is extracted three times successively using a 5:1, 3:1, and a 2:1 ratio of ethanol (containing 6% of water) to powder to extract the phospholipids. About 160 g of a waxy material comprising phospholipids as the major component with minor amounts of neutral lipids is obtained. A typical purified composition comprises:

| | |
|---|---|
| Polar lipids: | 82 wt % |
| PC content: | 72 wt % |
| esterfied HUFAs: | 33 wt % (of PC based on total lipids) |
| non polar (neutral) lipids: | 13 wt % |
| free fatty acids: | 5 wt % |
| Cholesterol/esters | 4 wt % |
| Iodine value: | 112 (range 110-130) |
| minerals, (ash) | 2 wt % |
| ethanol: | 2.2 wt % |
| water: | 1.9 wt % |

The MPL composition may be blended with 50 wt % of an oil which may be a fish oil, vegetable oil or medium chain glyceride such as Miglyol, to prepare a fluid lipophilic composition. Alternatively, it may also be hydrated overnight in glycerol at room temperature to prepare a hydophlic composition. The liquid compositions may be filled into soft gelatine capsules for oral use or it may be used in fish and larvae feeds as in Example 1.

### EXAMPLE 3

| | |
|---|---|
| *MPL with 72 wt % PC | 50 wt % |
| Glycerol 90% | 50 wt % |

The MPL is hydrated overnight in a solution of glycerol at room temperature to prepare a viscous gel like MPL concentrate.

### EXAMPLE 4

The MPL concentrate from Example 3 is mixed with up to 25 wt % lipophilic nutritional components which may be fish triglycerides and anti oxidants such as Vit E, ascorbyl palmitate, t-butylated hydroxytoluene, t-butylated hydroxyanisole, ascorbic acid or ethoxyquin. Additionally 25 wt % to 50 wt % of hydrophilic fish feed components such as fish protein hydrolysates may be added. 2 wt % sodium alginate in aqueous solution may be added to the MPL concentrate as shown in the example. The resultant aqueous suspension of lipid aggregates associated with nutritional components and alginate is used to prepare feed microparticles by cross linking the alginate in the composition in a bath containing Ca++ ions to prepare microparticles as described in WO0027218. The microparticles are recovered and dried. A typical composition is illustrated below.

| | |
|---|---|
| *MPL (with about 70 wt % PC) | 20.0 wt % |
| Glycerol (70 %) | 10.0 wt % |
| Water (containing 2% Sod alginate) 19.5 wt% | |
| Fish oil | 10.0 wt % |
| (lipophilic nutrional component) | |
| Fish protein hydrolysate | 40.0 wt % |
| (hydrophilic nutritional component) | |
| mixed antioxidants | 0.5 wt % |
| (tocopherol, ascorbylpalmitate) | |

*waxy MPL composition with a minimum PC content of about 40 wt % may also be used.

There is described marine phospholipids (MPL) compositions suitable for human, feed and aquaculture purposes comprising a dry composition comprising nutritional components selected from the group consisting of marine phospholipids, marine proteins and amino acid blends obtainable by fluid extraction of a dried marine raw material. The compositions have low amounts of neutral lipids and particularly low amounts of cholesterol and cholesterol esters. They may be used either as such in powder form and for preparing purified MPL compositions by ethanol extraction. Both forms may be used as nutritional supplements, ingredients for functional foods either on their own or in combination with biologically active compounds and to optimise the delivery and containment of nutritional components in multicomponent fish feed compositions.

There is further described a blend of the purified, ethanol extracted MPL compositions mixed with marine, vegetable, microbial or medium chain triglyceride oils to prepare liquid preparations for hard or soft gel encapsulation.

There is further described MPL concentrates comprising purified and clearly defined phospholipid types dispersed in hydrophilic medium which are nutritionally essential in larvae feed. Still further there is described MPL concentrates in unit dosage forms as supplements to supply highly bioavailable omega-3 fatty acids for human use, optionally with biologically active compounds.

1. A marine phospholipid (MPL) composition suitable for human, feed or aquaculture applications comprising a dry composition comprising nutritional components selected from the group consisting of marine phospholipids, marine proteins and amino acid blends obtainable by fluid extraction of a dried marine raw material.

2. The composition according to claim 1, wherein the dry composition is present in particulate or powdered form.

3. The composition according to claim 1, wherein the dry composition comprises a major amount of polar and a lower amount of non-polar (neutral) lipids.

4. The composition according to claim 1, wherein the dry composition comprises
I) 10 wt % to 30 wt % polar and neutral lipids; and
II) 70 wt % to 90 wt % marine proteins and amino acid blends.

5. The composition according to claim 1, wherein the dry composition comprises
I) 15 wt % to 30 wt % polar and neutral lipids;
II) 70 wt % to 85 wt % marine proteins, amino acid blend and minerals.

6. The composition according to claim 4, wherein the total lipid content consists of 70 wt % to 95 wt % polar lipids and 5 wt % to 30 wt % consist of neutral lipids.

7. The composition according to claim 4, wherein the total lipid content consists of 80 wt % to 95 wt % polar lipids and 5 wt % to 20 wt % consist of neutral lipids.

8. The composition according to claim 4, wherein 40 wt % to 80 wt % of the polar lipids consist of phosphatidylcholine and their monoacyl derivative, wherein said phosphatidylcholine and their monoacyl derivative are esterified with 30 wt % to 60 wt % HUFAs.

9. The composition according to claim 4, wherein 20 wt % to 60 wt % of the polar lipids consist of phospholipids selected from the group consisting of phosphatidyl ethanolamine, phosphatidyl inositol, phosphatidyl serine, sphingomyelin and the monoacyl derivatives thereof.

10. The composition according to claim 4, which is prepared by means of ethanol extraction to achieve a waxy constitution.

11. The composition according to claim 10 comprising
i) 70 wt % to 95 wt % of polar and neutral lipids,
ii) 5 wt % to 30 wt % of neutral lipids.

12. The composition according to claim 11 blended with 30 wt % to 60 wt % of a fish oil or any other edible neutral oil for use in hard or soft gelatine capsules.

13. A homogeneous water dispersible MPL concentrate comprising,
i) 25 wt % to 75 wt % marine phospholipids;
ii) 15 wt % to 75 wt % of ethanol or at least one polyol or mixtures thereof.
iii) water to make 100%; and,
iv) optionally further additives selected from the group consisting of polymers, nutritional components, stabilizers, preservatives and antioxidants.

14. A composition according to claim 1 mixed homogeneously with phospholipids selected from the group consisting of soya, plant, egg, semi-synthetic, enzyme modified phospholipids and optionally biologically active compounds.

15. A method for further processing the MPL composition according to claim 1, which comprises separating the marine proteins and amino acid blends from the dry composition.

16. A method for further processing the water dispersible MPL concentrate according to claim 13, which comprises dispersing the concentrate in water and administering the dispersion to fish and/or larvae.

17. A process for preparing a waxy marine phospholipid (MPL) composition suitable for human or aquaculture applications, which process comprises the step of preparing a dry composition comprising nutritional components selected from the group consisting of marine phospholipids, marine proteins, amino acid and minerals blend by subjecting a dried marine raw material to fluid extraction.

18. A process for preparing a marine phospholipid composition according to claim 1, which comprises preparing the dry composition by subjecting the dried marine raw material to fluid extraction with acetone.

19. A process for preparing a marine phospholipid composition according to claim 1, which process comprises preparing the dry composition by subjecting the dried marine raw material to fluid extraction with supercritical gas.

20. A process for preparing a water dispersible MPL concentrate from an MPL composition involving fluid and ethanol extraction.

21. A composition according to claim 10 mixed homogeneously with phospholipids selected from the group consisting of soya, plant, egg, semi-synthetic, enzyme modified phospholipids and optionally biologically active compounds.
